(19) United States
(12) Patent Application Publication
(10) Pub. No.: US 2013/0034909 A1
(43) Pub. Date: Feb. 7, 2013

(54) DIAGNOSIS METHOD AND DIAGNOSIS APPARATUS FOR OIL-FILLED ELECTRICAL APPARATUS

(71) Applicants: Satoru Toyama, Chiyoda-ku (JP); Junji Tanimura, Chiyoda-ku (JP); Fukutaro Kato, Chiyoda-ku (JP); Tsuyoshi Amimoto, Chiyoda-ku (JP); Eiichi Nagao, Chiyoda-ku (JP); Takeshi Kawashima, Chiyoda-ku (JP); Noboru Hosokawa, Chiyoda-ku (JP)

(72) Inventors: Satoru Toyama, Chiyoda-ku (JP); Junji Tanimura, Chiyoda-ku (JP); Fukutaro Kato, Chiyoda-ku (JP); Tsuyoshi Amimoto, Chiyoda-ku (JP); Eiichi Nagao, Chiyoda-ku (JP); Takeshi Kawashima, Chiyoda-ku (JP); Noboru Hosokawa, Chiyoda-ku (JP)

(73) Assignee: Mitsubishi Electric Corporation, Tokyo (JP)

(21) Appl. No.: 13/640,509

(22) PCT Filed: May 10, 2011
(86) PCT No.: PCT/JP2011/060768

§ 371 Date: Oct. 11, 2012

(30) Foreign Application Priority Data
Jun. 2, 2010 (JP) 2010-127031
Mar. 25, 2011 (JP) PCT/JP2011/057388

(51) Int. Cl. G01N 33/20; G01N 31/00

(52) U.S. Cl. 436/80; 436/120; 436/131

(57) ABSTRACT

Provided is a diagnosis method for an oil-filled electrical apparatus, which is for evaluating the degree of danger of copper sulfide being generated within the oil-filled electrical apparatus, comprising: a first step for detecting a specific compound contained within insulating oil inside the oil-filled electrical apparatus; a second step for evaluating the possibility of copper sulfide being generated inside the oil-filled electrical apparatus, on the basis of the result detected by the first step; and a third step for diagnosing the degree of danger of a malfunction occurring in the oil-filled electrical apparatus, on the basis of the evaluation result obtained in the second step. The specific compound contains dibenzyl disulfide and/or a reaction product of a radical resulting from dibenzyl disulfide, and di-tert-butyl-p-cresol and/or a reaction product of a radical resulting from di-tert-butyl-p-cresol, or, di-tert-butyl-phenol and/or a reaction product of a radical resulting from di-tert-butyl-phenol.

### TECHNICAL FIELD

The present invention relates to anomaly diagnosis as to sulfidation corrosion of a transformer, and more detailedly, it relates to a technique of predicting generation of copper sulfide onto insulating paper.

### BACKGROUND ART

Sulfidation corrosion is such a phenomenon that conductive copper sulfide is generated by reaction between copper which is the material for a transformer and a sulfur component in insulating oil. This copper sulfide is a semiconductor, and hence insulating performance lowers when the same adheres to an insulator. Insulating paper is used for coil insulation particularly in a large-sized transformer or the like, and hence a short circuit takes place between coils when copper sulfide adheres to this insulating paper which is an insulator, and the transformer is broken. However, the details of the generation mechanism thereof are unknown, and there has existed no effective diagnosis technique for an existing transformer.

The inventors have recently diligently studied a reaction mechanism related to copper sulfide generation, to recognize that such reaction that dibenzyl disulfide adsorbs to a copper plate takes place as a first stage, such reaction that the dibenzyl disulfide reacts with copper to generate a dibenzyl disulfide-Cu complex takes place as a second stage, and such reaction that the dibenzyl disulfide-Cu complex decomposes into a benzyl radical and a benzyl sulfenyl radical and copper sulfide takes place as a third stage (NPL 1: S. Toyama, J. Tanimura, N. Yamada, E. Nagao, T. Amimoto, “Highly Sensitive Detection Method of Dibenzyl Disulfide and the Elucidation of the Mechanism of Copper Sulfide Generation in Insulating Oil”, April 2009, IEEE TDEI Vol. 16, No. 2, pp. 509-515).

When using the technique of NPL 1, insulating oil can be collected from an existing transformer in operation, and the quantity of copper sulfide generated in the existing transformer can be predicted. A benzyl radical and a benzyl sulfenyl radical react with each other respectively and mutually, and bibenzyl, dibenzyl sulfide and dibenzyl disulfide generate as byproducts. Information related to generation of copper sulfide can be obtained by analysis of these byproducts.

However, whether or not adhesion of copper sulfide reaches insulating paper has not been predictable even with this technique (PTL 1: Japanese Patent Laying-Open No. 2010-10439).

There is absolutely no technique of predicting generation of copper sulfide except for the aforementioned technique, and an operation of reducing a risk of copper sulfide generation is generally performed by analyzing dibenzyl disulfide which is a causative agent of copper sulfide by analysis of oil, collecting insulating oil in an existing transformer when this compound is detected and exchanging the same for new insulating oil or removing the dibenzyl disulfide by treating the collected insulating oil and thereafter returning the treated insulating oil into the transformer (PTL 2: U.S. Patent Laying-Open No. 2008/0251424).

There are a large number of techniques predicting inconvenience of a transformer other than generation of copper sulfide. Further, there are a large number of methods diagnosing abnormal overheat or abnormal discharge by detecting a certain type of compound. According to these methods, it is assumed that soundness of a transformer can be diagnosed by analyzing a volatile component in oil generated due to deterioration of insulating paper or the like.

For example, there is known an anomaly diagnosis method in an oil-filled electrical apparatus characterized in diagnosing abnormal overheat or abnormal discharge in the apparatus through the presence or absence of detection of acetic acid, 3-pentanone, 2,5-dimethylfuran, butyl aldehyde, 2-methoxy ethanol, methanethiol, dimethyl sulfide, ammonia, 1,3-diazine, methyl vinyl acetylene or 2-methyl-1,3-butadiene not detected from an ordinary oil-filled electrical apparatus in operation but easily detectable only in overheat or abnormal discharge (PTL 3: Claim 2 etc. of Japanese Patent Laying-Open No. 9-72892).

There are many cases where an anomaly of an oil-filled electrical apparatus results from dielectric breakdown of a coil or the like caused by adhesion of copper sulfide to insulating paper, and hence it is particularly important to predict adhesion of copper sulfide to an insulating paper surface in diagnosis of the oil-filled electrical apparatus. However, although the quantity or the like of copper sulfide generated in the oil-filled electrical apparatus e can be predicted when using the invention of NPL 1 or PTL 1, whether or not the generated copper sulfide adheres to insulating paper cannot be determined. Although the invention of PTL 2 is also a technique related to copper sulfide generation in a transformer, the same is not a technique allowing determination as to whether or not copper sulfide is generated on an insulating paper surface. Although the invention of PTL 3 is a technique of diagnosing an anomaly of a transformer, the same does not allow determination of generation of copper sulfide.

### CITATION LIST

### Patent Literature

PTL 1: Japanese Patent Laying-Open No. 2010-10439
PTL 2: U.S. Patent Laying-Open No. 2008/0251424
PTL 3: Japanese Patent Laying-Open No. 9-72892

### Non Patent Literature

NPL 1: S. Toyama, J. Tanimura, N. Yamada, E. Nagao, T. Amimoto, “Highly Sensitive Detection Method of Dibenzyl Disulfide and the Elucidation of the Mechanism of Copper Sulfide Generation in Insulating Oil”, April 2009, IEEE TDEI Vol. 16, No. 2, pp. 509-515

### SUMMARY OF INVENTION

### Technical Problem

The present invention has been proposed in order to solve the aforementioned problems, and aims at providing a diagnosis method for an oil-filled electrical apparatus capable of determining whether or not copper sulfide is generable on an insulating paper surface of a coil or the like dipped in insulating oil in the oil-filled electrical apparatus.

### Solution to Problem

The present invention provides a diagnosis method for an oil-filled electrical apparatus evaluating a risk of copper sulfide generation in the oil-filled electrical apparatus, including:

a first step of detecting a specific compound contained in insulating oil in the said oil-filled electrical apparatus,

a second step of evaluating a possibility of copper sulfide generation in the said oil-filled electrical apparatus on the basis of the result of detection obtained in the said first step, and

a third step of diagnosing a risk of anomaly occurrence in the said oil-filled electrical apparatus on the basis of the result of evaluation obtained in the said second step, in which

the said specific compound contains dibenzyl disulfide and/or a reaction product of a radical resulting from dibenzyl disulfide, and

di-tert-butyl-p-cresol and/or a reaction product of a radical resulting from di-tert-butyl-p-cresol, or, di-tert-butyl-phenol and/or a reaction product of a radical resulting from di-tert-butyl-phenol.

Preferably, the said specific compound contains dibenzyl sulfide, and di-tert-butyl-p-cresol or di-tert-butyl-phenol.

Preferably, the reaction product of the radical resulting from the said dibenzyl disulfide is a compound of at least one type selected from a group consisting of benzaldehyde, benzyl alcohol, bibenzyl, dibenzyl sulfide and dibenzyl sulfoxide.

Preferably, the said specific compound contains a reaction product of a radical resulting from dibenzyl disulfide and a radical resulting from di-tert-butyl-p-cresol, or, a reaction product of a radical resulting from dibenzyl disulfide and a radical resulting from di-tert-butyl-phenol.

Preferably, the method evaluates in the said second step that the possibility of copper sulfide generation in the said oil-filled electrical apparatus is high in a case where the said specific compound is detected in the said first step, and

diagnoses in the said third step that the risk of anomaly occurrence in the said oil-filled electrical apparatus is high in a case where the possibility of copper sulfide generation is evaluated as high in the said second step.

Preferably, copper sulfide generation in the said oil-filled electrical apparatus is copper sulfide generation on a surface of insulating paper.

The present invention also relates to a diagnosis apparatus for an oil-filled electrical apparatus for evaluating a risk of copper sulfide generation in the oil-filled electrical apparatus, including:

a detection unit detecting a specific compound contained in insulating oil in the said oil-filled electrical apparatus,

an evaluation unit evaluating a possibility of copper sulfide generation in the said oil-filled electrical apparatus on the basis of the result of detection obtained in the said detection unit, and

a diagnosis unit diagnosing a risk of anomaly occurrence in the said oil-filled electrical apparatus on the basis of the result of evaluation obtained in the said evaluation unit, in which

the said specific compound contains dibenzyl disulfide and/or a reaction product of a radical resulting from dibenzyl disulfide, and

di-tert-butyl-p-cresol and/or a reaction product of a radical resulting from di-tert-butyl-p-cresol, or, di-tert-butyl-phenol and/or a reaction product of a radical resulting from di-tert-butyl-phenol.

### Advantageous Effects of Invention

According to the inventive diagnosis method, such a nonconventional remarkable effect is attained that a risk of copper sulfide generation on an insulating paper surface of a coil or the like dipped in insulating oil in an oil-filled electrical apparatus can be correctly evaluated by detecting (measuring) not only dibenzyl disulfide but also di-tert-butyl-p-cresol or di-tert-butyl-phenol in analysis of insulating oil collected from an existing oil-filled electrical apparatus (transformer or the like).

### BRIEF DESCRIPTION OF DRAWINGS

FIG. 1 is a graph showing correlation between the quantities of reduction of DBDS and the total quantities of reaction products of radicals resulting from DBDS.

FIG. 2A is an example of a radical resulting from DBPC.

FIG. 2B is another example of a radical resulting from DBPC.

FIG. 3 is an example of a compound having molecular weight of 310 resulting from reaction between a benzyl radical and DBPC.

FIG. 4 is a block diagram of a diagnosis apparatus for an oil-filled electrical apparatus according to a fourth embodiment.

FIG. 5 is a sectional view showing a structural example of the oil-filled electrical apparatus shown in FIG. 4.

FIG. 6 is a plan view showing one of a plurality of winding layers constituting a coil.

FIG. 7 is a sectional view showing a section of the wiring layer shown in FIG. 6 along the line A-A.

FIG. 8 is a graph showing test results of a Test Example 3.

### DESCRIPTION OF EMBODIMENTS

As hereinabove described, it is recognized that such reaction that dibenzyl disulfide (DBDS) adsorbs to a copper plate takes place as a first stage, such reaction that DBDS reacts with copper to generate a DBDS-Cu complex takes place as a second stage, and such reaction that the DBDS-Cu complex decomposes into a benzyl radical and a benzyl sulfenyl radical and copper sulfide takes place as a third stage as the mechanism of copper sulfide generation.

Among these compounds participating in the copper sulfide generation mechanism, only the copper sulfide is insoluble in insulating oil. When copper sulfide is generated on a copper plate surface, therefore, there is no possibility that this copper sulfide transfers to insulating paper.

On the other hand, the DBDS-Cu complex exhibits oil solubility since the same is a complex, and hence the same transfers from the copper plate surface to the insulating paper, and can be adsorbed to the insulating paper surface. In a case where such reaction takes place that this DBDS-Cu complex decomposes into a benzyl radical and a benzyl sulfenyl radical and copper sulfide on the insulating paper surface, copper sulfide is generated on the insulating paper surface.

Further, when another radical is present in the vicinity of a sulfur element in such an instant that the DBDS-Cu complex radically decomposes on the copper plate surface, it is predicted that the sulfur element and another radical radically combine with each other to return to the complex. Thus, the quantity of the copper sulfide generated on the copper plate surface decreases and the quantity of the oil-soluble complex increases, and hence it is conceivable that the quantity of generation of the copper sulfide on the insulating paper surface increases. In a case where another radical other than a radical deriving from DBDS coexists in insulating oil, therefore, it is predicted that the quantity of generation of copper sulfide on an insulating paper surface increases.

It is necessary that such another radical is present in the vicinity of the sulfur element stably and in a large quantity, and a radical-based antioxidant such as di-tert-butyl-p-cresol or di-tert-butyl-phenol (DBP) is conceivable, for example. The diagnosis method according to the present invention is characterized in detecting di-tert-butyl-p-cresol and/or a reaction product of a radical resulting from di-tert-butyl-p-cresol (or, di-tert-butyl-phenol and/or a reaction product of a radical resulting from di-tert-butyl-phenol). In the present invention, di-tert-butyl-p-cresol is preferably 2,6-di-tert-butyl-p-cresol (DBPC).

DBDS, DBPC and DBP are detectable according to an existing technique. The same can be determined up to 0.1 ppmw when employing a gas chromatograph/mass spectrometer, for example.

### First Embodiment

An embodiment of the diagnosis method for an oil-filled electrical apparatus according to the present invention is described. The diagnosis method according to this embodiment is a diagnosis method for an oil-filled electrical apparatus evaluating a risk of copper sulfide generation on an insulating paper surface of a coil or the like in the oil-filled electrical apparatus, and includes:

a first step of detecting a specific compound contained in insulating oil in the oil-filled electrical apparatus,

a second step of evaluating a possibility of copper sulfide generation in the said oil-filled electrical apparatus on the basis of the result of detection obtained in the said first step, and

a third step of diagnosing a risk of anomaly occurrence in the said oil-filled electrical apparatus on the basis of the result of evaluation obtained in the said second step.

More specifically, the possibility of copper sulfide generation in the oil-filled electrical apparatus is evaluated as high in the second step in a case where the said specific compound is detected in the first step. In the third step, it is diagnosed that the risk of anomaly occurrence in the oil-filled electrical apparatus is high in a case where the possibility of copper sulfide generation is evaluated as high in the second step.

In this embodiment, DBPC and DBDS are detected (measured) as specific compounds.

According to this embodiment, it becomes possible to correctly evaluate a risk of copper sulfide generation on an insulating paper surface of a coil or the like dipped in insulating oil in an oil-filled electrical apparatus by detecting (measuring) not only DBDS but also DBPC in analysis of insulating oil collected from an existing oil-filled electrical apparatus (transformer or the like).

### Second Embodiment

This embodiment is a diagnosis method different from the first embodiment in a point of measuring DBPC and a reaction product of a radical resulting from DBDS as the aforementioned specific compounds, and the remaining points are similar to those of the first embodiment.

DBDS and DBPC in insulating oil are gradually consumed due to use of the insulating oil in a transformer. Even in insulating oil having contained DBPC and DBDS before use, therefore, there is a case where DBDS and DBPC are not detected in a case of measuring only DBDS and DBPC after long-term use of the insulating oil. In such a case, there is a possibility that the transformer, which must be diagnosed as being at a high risk, is erroneously diagnosed as being at a low risk.

This embodiment can avoid such an erroneous determination by detecting a compound (reaction product of the radical resulting from DBDS) generated by consumption of DBDS in place of measurement of DBDS.

For example, benzaldehyde, benzyl alcohol, bibenzyl, dibenzyl (mono)sulfide and dibenzyl sulfoxide can be listed as reaction products of radicals resulting from DBDS. These compounds can be analyzed by employing gas chromatograph/mass spectrometry, for example.

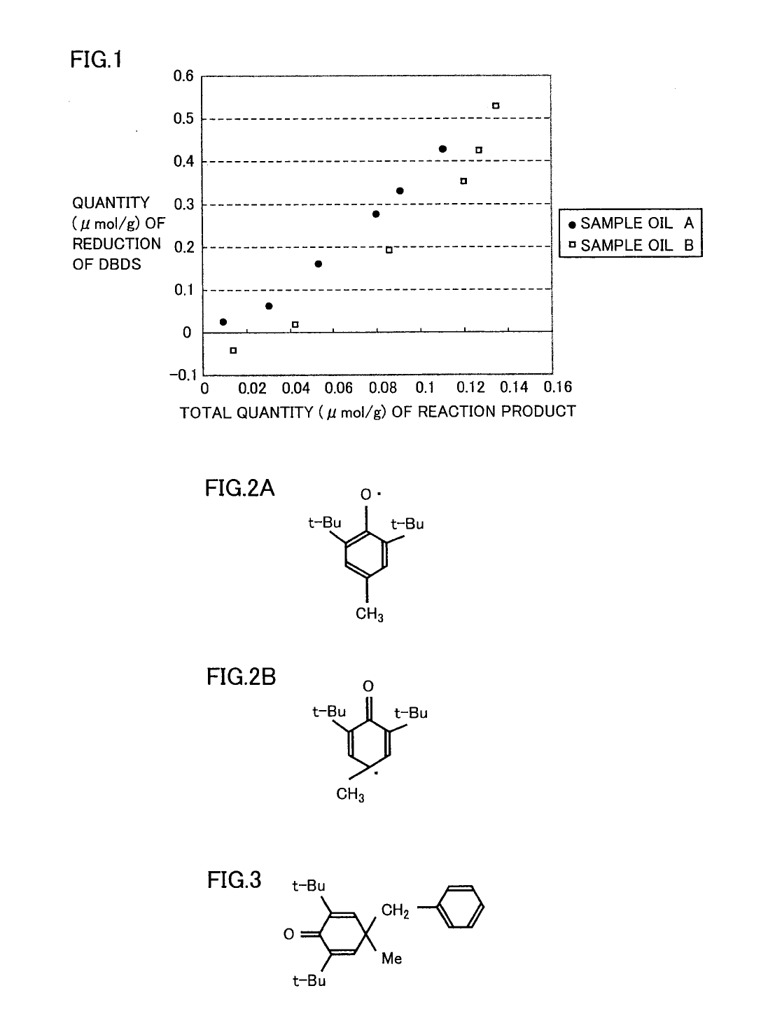

FIG. 1 shows the relation between the quantities of reduction (quantities of consumption) of DBDS and the total quantities of these reaction products (benzaldehyde, benzyl alcohol, bibenzyl, dibenzyl (mono)sulfide and dibenzyl sulfoxide) in sample oil A and sample oil B in Test Example 1 described later. As shown in FIG. 1, the total quantities of these reaction products and the quantities of reduction of benzyl sulfide are correlated with each other. Therefore, it is determinable that DBDS has been added if these compounds are detected also in a case where DBDS is not detected.

These compounds are correlated with the quantity of reduction of DBDS also as individual contents, and hence it is provable that DBDS has been present without employing the total quantity, by measuring at least one of these compounds.

As to detection of DBPC, whether or not DBPC has been added to electric insulating oil at the time of start of use can be determined by a method of analyzing a reaction product of DBPC and a peroxy radical by a proper analytical method with a gas chromatograph mass spectrometer, for example. It is widely known that DBPC easily becomes a radical shown in FIG. 2A or 2B and reacts with a radical such as a peroxy radical.

### Third Embodiment

This embodiment is a diagnosis method different from the first and second embodiments in a point of measuring a reaction product of a radical resulting from DBPC and a radical resulting from DBDS as the aforementioned specific compound, while the remaining points are similar to those of the first and second embodiments.

The measurement of the reaction product of the radical resulting from DBPC and the radical resulting from DBDS corresponds to measurement of “a specific compound containing a reaction product of a radical resulting from dibenzyl disulfide and a reaction product of a radical resulting from di-tert-butyl-p-cresol” in the diagnosis method according to the present invention. In other words, the detection of “the specific compound containing the reaction product of the radical resulting from dibenzyl disulfide and the reaction product of the radical resulting from di-tert-butyl-p-cresol” includes not only a case of detecting both of a reaction product of radicals resulting from dibenzyl disulfide and a reaction product of radicals resulting from di-tert-butyl-p-cresol, but also a case of detecting a reaction product of a radical resulting from dibenzyl disulfide and a radical resulting from di-tert-butyl-p-cresol.

When analyzing electric insulating oil with a gas chromatograph/mass spectrometer (GC/MS) in a case where DBPC and DBDS have been added at the same time, a compound whose molecular weight is 310 is detected. It is conceivable that this compound whose molecular weight is 310 is 4-benzyl-2,6-di-tert-butyl-4-methyl-2,5-cyclohexadienone (whose structural formula is shown in FIG. 3), for example. This compound is a reaction product of a radical resulting from DBPC and a benzyl radical which is a radical resulting from DBDS. With only detection of this compound, a risk of generation of copper sulfide in an oil-filled electrical apparatus can be evaluated similarly to the first embodiment detecting DBPC and DBDS.

According to the diagnosis method of this embodiment, it is determinable that there is a high risk that copper sulfide adheres to insulating paper in an oil-filled electrical apparatus similarly to the first embodiment, also when DBDS and DBPC have been consumed due to generation of copper sulfide or the like as a result of long-term use of insulating oil. Further, the oil-filled electrical apparatus can be simply diagnosed with only measurement of one type of compound.

In the diagnosis method according to the present invention, a risk of copper sulfide generation in an oil-filled electrical apparatus may be evaluated by detecting DBDS and DBPC in insulating oil and further detecting a reaction product, such as 4-benzyl-2,6-di-tert-butyl-4-methyl-2,5-cyclohexadienone, of a radical resulting from DBDS and a radical resulting from DBPC, for example. In this case, the concentrations of DBDS and DBPC in the insulating oil before use can be more correctly estimated, whereby the risk of copper sulfide generation on an insulating paper surface in the oil-filled electrical apparatus can be more correctly evaluated.

Also in a case of regarding DBP as a detection object in place of DBPC in the aforementioned first to third embodiments, it is possible to correctly evaluate a risk of copper sulfide generation on an insulating paper surface of a coil or the like dipped in insulating oil in an oil-filled electrical apparatus.

### Fourth Embodiment

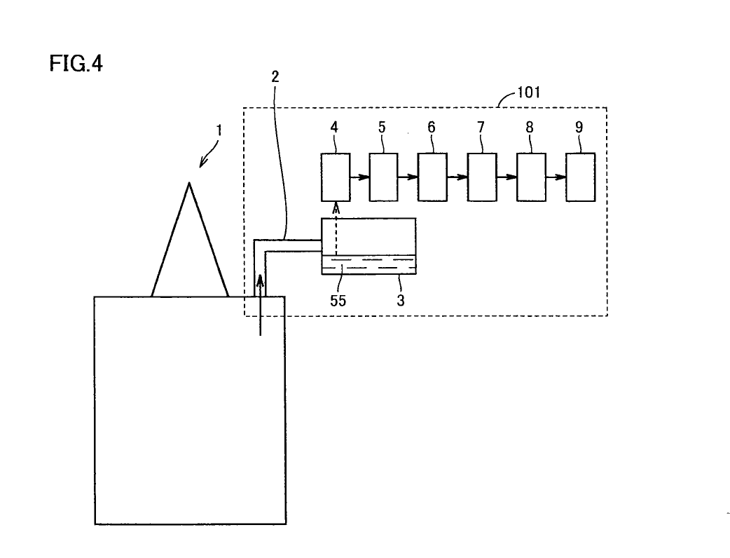

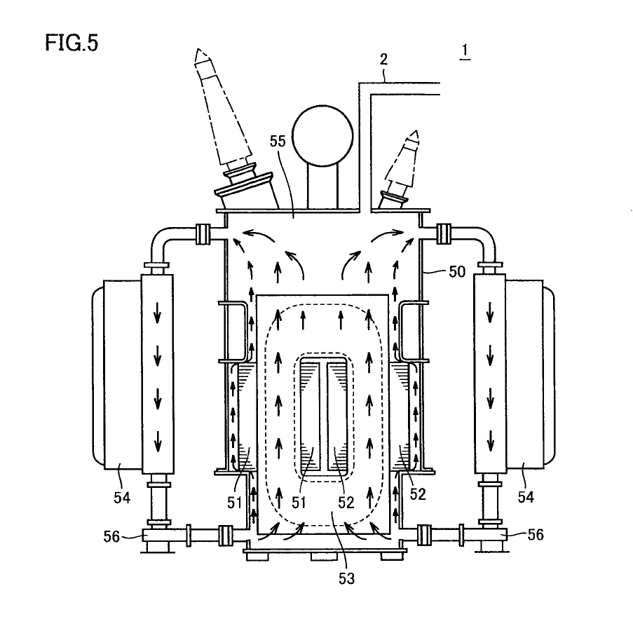

This embodiment is an embodiment of a diagnosis method for implementing the diagnosis methods for oil-filled electrical apparatuses according to the aforementioned embodiments. FIG. 4 shows a block diagram of a diagnosis apparatus for an oil-filled electrical apparatus according to this embodiment. Referring to FIG. 4, a diagnosis apparatus 101 includes a pipe 2, a tank 3, an oil collector 4, a pretreater 5, a detection unit 6, an evaluation unit 7, a diagnosis unit 8 and a display 9.

FIG. 5 is a sectional view showing a structural example of an oil-filled electrical apparatus shown in FIG. 4. Referring to FIG. 5, an oil-filled electrical apparatus 1 is a transformer, for example, and includes a tank 50, iron cores 51 and 52, a coil 53, coolers 54 and insulating oil 55.

Iron cores 51 and 52 and coil 53 are stored in tank 50. Coil 53 is enclosed by iron cores 51 and 52. The inner portion of tank 50 is filled with insulating oil 55. Therefore, coil 53 is dipped in insulating oil 55.

Insulating oil 55 circulates in oil-filled electrical apparatus 1 by pumps 56. As shown by arrows in FIG. 5, insulating oil 55 goes out of tank 50, and is cooled by coolers 54. Cooled insulating oil 55 returns to tank 50. Insulating oil 55 is mineral oil or synthetic oil, for example.

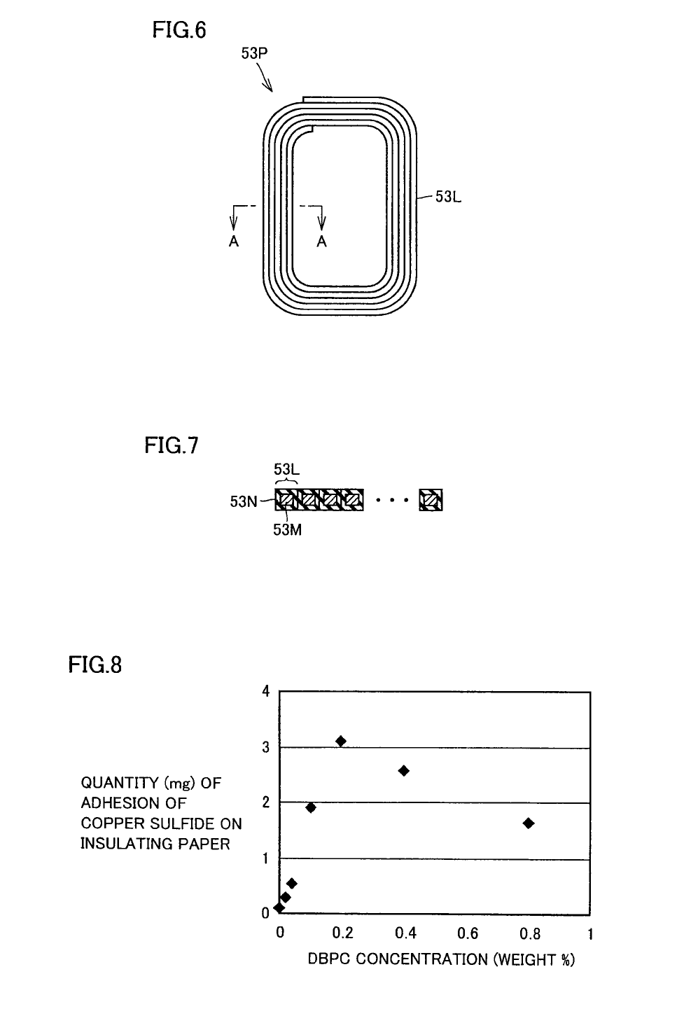

Coil 53 is constituted of a plurality of winding layers stacked in one direction. FIG. 6 is a plan view showing one of the plurality of winding layers constituting the coil. FIG. 7 is a sectional view showing a section of the winding layer shown in FIG. 6 along the line A-A.

Referring to FIGS. 6 and 7, a winding layer 53P is constituted of a paper-wrapped conductor 53L. Paper-wrapped conductor 53L is spirally wound in the same plane. Paper-wrapped conductor 53L has a conductor 53M containing copper and insulating paper 53N covering conductor 53M. Insulating paper 53N contains cellulose molecules.

Referring to FIG. 4, tank 3 is connected to oil-filled electrical apparatus 1 by pipe 2. When insulating oil 55 is collected from inside oil-filled electrical apparatus 1, part of the insulating oil in oil-filled electrical apparatus 1 passes through pipe 2, and flows into tank 3. Oil collector 4 is a pump, for example, and collects the insulating oil in tank 3. The insulating oil in tank 3 is employed for detection of specific compounds with detection unit 6. Pretreater 5 performs pretreatment of the insulating oil before the insulating oil in tank 3 is transmitted to detection unit 6. In detection unit 6, residual concentrations of the aforementioned specific compounds are measured.

Evaluation unit 7 and diagnosis unit 8 are constituted of computers, for example, and execute arithmetic processing on the basis of maps and programs stored therein. More specifically, evaluation unit 7 receives measured values of the specific compounds from detection unit 6, and evaluates a possibility of copper sulfide generation in the oil-filled electrical apparatus. Diagnosis unit 8 receives the result of evaluation of evaluation unit 7, and diagnoses a risk of anomaly occurrence in the oil-filled electrical apparatus.

Display 9 displays the result of diagnosis by diagnosis unit 8, i.e., the risk of anomaly occurrence in the oil-filled electrical apparatus on an unshown screen. Thus, it becomes possible to grasp the result of diagnosis by diagnosis apparatus 101.

### EXAMPLES

Results of a test of confirming the relation between the quantities of compounds contained in insulating oil deriving from dibenzyl disulfide; and di-tert-butyl-p-cresol or di-tert-butyl-phenol, and generation of copper sulfide on insulating paper surfaces are now shown in relation to the diagnosis method according to the present invention.

### Test Example 1

First, naphthene-based transformer oil (sample oil A) confirmed as containing no corrosive sulfur under ASTM D 1275B is prepared. Then, dibenzyl disulfide is added to this transformer oil by a prescribed quantity. The same was added by 100 ppmw (w/w) in this experiment. This oil is regarded as sample oil B. Oil prepared by adding DBPC to the sample oil B by 0.4 weight % (w/w) is regarded as sample oil C.

A test related to generation of copper sulfide is conducted by a method according to IEC 62535 of IEC (International Electrotechnical Commission) standards by employing these insulating oil samples. 15 grams of this transformer oil and a copper plate (30 mm by 7.5 mm by 1.5 mm) wrapped with one layer of kraft paper are sealed in a bottle having an internal volume of 30 cc, covered with a silicone rubber stopper, and thereafter heated under conditions of 150° C. and 24 to 144 hours.

Table 1 shows the relation between copper sulfide adhesion situations on insulating paper surfaces after the test and heating times. The numerical values in the table are employed in the following meanings:

1: no adhesion of copper sulfide

2: slightly adhered to end portion of insulating paper

3: adhered in wider range than 2

4: adhered to whole surface

TABLE 1

| | 1^{st} Day | 2^{nd} Day | 3^{rd} Day | 4^{th} Day | 5^{th} Day | 6^{th} Day |
|---|---|---|---|---|---|---|
| Sample Oil A | 1 | 1 | 1 | 1 | 1 | 1 |
| Sample Oil B | 1 | 2 | 2 | 2 | 2 | 2 |
| Sample Oil C | 3 | 3 | 3 | 3 | 4 | 4 |

As shown in Table 1, no dibenzyl sulfide serving as the raw material for copper sulfide is added and hence no copper sulfide is generated in sample oil A. In sample oil B, generation of copper sulfide on an insulating paper surface is observed. As a result of obtaining the quantity of adhesion of copper sulfide by analysis, the same was 1.9 (1^{st} day) to 9.5 μg/cm² (6^{th} day). In sample oil C, remarkable generation of copper sulfide on an insulating paper surface is observed. As a result of obtaining the quantity of adhesion of copper sulfide by analysis, the same was 48 (1^{st} day) to 47.7 μg/cm² (6^{th} day).

Therefore, a case of employing sample oil C can be regarded as being at a high risk of at least about five times as compared with a case of employing sample oil B. In other words, a transformer employing insulating oil to which DBDS and DBPC are added can be determined as being at a high risk (having a high risk of anomaly occurrence).

### Test Example 2

The following investigation was conducted by the same method as Test Example 1, except that paraffin-based mineral oil was used in place of the naphthene-based mineral oil:

Paraffin-based transformer oil (sample oil D) confirmed as containing no corrosive sulfur under ASTM D 1275B is prepared. Then, oil prepared by adding dibenzyl disulfide to this transformer oil (sample oil D) by 100 ppmw is regarded as sample oil E. Oil prepared by adding DBPC to sample oil E by 0.4 weight % is regarded as sample oil F.

A test related to generation of copper sulfide was conducted by employing these insulating oil samples by a method according to IEC 62535. 15 grams of this transformer oil and a copper plate (30 mm by 7.5 mm by 1.5 mm) wrapped with one layer of kraft paper are sealed in a bottle having an internal volume of 30 cc, covered with a silicone rubber stopper, and thereafter heated under conditions of 150° C. and 24 to 144 hours.

Table 2 shows the relation between copper sulfide adhesion situations on insulating paper surfaces after the test and heating times. The numerical values in the table are employed in meanings similar to those in Table 1.

TABLE 2

| | 1^{st} Day | 2^{nd} Day | 3^{rd} Day | 4^{th} Day | 5^{th} Day | 6^{th} Day |
|---|---|---|---|---|---|---|
| Sample Oil D | 1 | 1 | 1 | 1 | 1 | 1 |
| Sample Oil E | 2 | 2 | 2 | 2 | 2 | 2 |
| Sample Oil F | 1 | 3 | 3 | 3 | 4 | 4 |

As shown in Table 2, no dibenzyl disulfide serving as the raw material for copper sulfide is added and hence no copper sulfide is generated in sample oil D. In sample oil E, generation of copper sulfide on an insulating paper surface is observed. As a result of analyzing the quantity of adhesion of copper sulfide, the quantity of adhesion was 3.5 μg/cm² (1^{st} i day) to 10.7 μg/cm² (6^{th} day). In sample oil F, remarkable generation of copper sulfide on an insulating paper surface is observed. As a result of analyzing the quantity of adhesion of copper sulfide, the quantity of adhesion was 4.8 μg/cm² (1^{st} day) to 53.1 μg/cm² (6^{th} day).

Therefore, a case of employing sample oil F can be regarded as being at a high risk of about five times with respect to a case of employing sample oil D. In other words, a transformer employing insulating oil to which DBDS and DBPC are added can be determined as being at a high risk.

### Test Example 3

In Test Examples 1 and 2, the experiments were conducted while fixing the concentrations of DBPC to 0.4 weight %. In this Test Example, a test related to generation of copper sulfide similar to that in Test Example 1 was conducted while setting concentrations of DBPC to 0, 0.02, 0.04, 0.1, 0.2, 0.4 and 0.8 weight % and setting concentrations of DBDS to 100 ppmw in order to clarify influence exerted by the concentrations of DBPC on quantities of copper sulfide on insulating paper surfaces, and quantities of adhesion of copper sulfide on insulating paper were measured. FIG. 8 shows a graph plotting the quantities of adhesion of copper sulfide on insulating paper with respect to the concentrations of DBPC.

As shown in FIG. 8, the quantity of adhesion of copper sulfide on insulating paper clearly increases as compared with an unadded case, even if the concentration of DBPC is 0.02 weight %. The quantities of copper sulfide on the insulating paper surfaces increased following the increase of the DBPC concentrations up to the DBPC loading of 0.2 weight %, and the quantities of copper sulfide on insulating paper thereafter decreased from the maximum value of 0.2 weight % following the increase of the concentrations of DBPC.

### Test Example 4

In Test Examples 1 to 3, the experiments employing DBPC as antioxidants were conducted. In this Test Example, an experiment employing DBP (di-tert-butyl-phenol) as antioxidants was conducted. In other words, an experiment similar to that in Test Example 1 was conducted as to naphthene-based transformer oil (sample oil A) similar to that in Test Example 1 and oil (sample oil G) prepared by adding 0.4 weight % of DBP and 100 ppmw of DBDS to sample oil A.

Table 3 shows the relation between copper sulfide adhesion situations on insulating paper surfaces after the test and heating times. Numerical values in the table are employed in meanings similar to those in Table 1.

TABLE 3

| | 1^{st} Day | 2^{nd} Day | 3^{rd} Day | 4^{th} Day | 5^{th} Day | 6^{th} Day |
|---|---|---|---|---|---|---|
| Sample Oil A | 1 | 1 | 1 | 1 | 1 | 1 |
| Sample Oil G | 3 | 3 | 3 | 3 | 3 | 3 |

As shown in Table 3, the quantities of copper sulfide adhering to the insulating paper surfaces were small not to reach full-scale adhesion in the case of DBP as compared with the case of DBPC, while the quantities of adhesion of copper sulfide on the insulating paper surfaces clearly increased as compared with the case (sample oil B in Table 1) where no antioxidant was added. Therefore, a transformer employing insulating oil to which DBP is added along with DBDS can be determined as being at a higher risk than a transformer employing insulating oil to which neither DBP nor DBPC is added.

The embodiments and Examples disclosed this time must be considered as illustrative in all points and not restrictive. The range of the present invention is shown not by the above description but the scope of claims for patent, and it is intended that all modifications within the meaning and range equivalent to the scope of claims for patent are included.

### REFERENCE SIGNS LIST

1 oil-filled electrical apparatus, 2 pipe, 3, 50 tank, 6 detection unit, 7 evaluation unit, 8 diagnosis unit, 9 display, 51, 52 iron core, 53 coil, 53L paper-wrapped conductor, 53M conductor, 53N insulating paper, 53P winding layer, 54 cooler, 55 insulating oil, 56 pump, 101 diagnosis apparatus.

1: A diagnosis method, comprising:
(a) detecting a specific compound contained in insulating oil in an oil-filled electrical apparatus;
(b) evaluating a possibility of copper sulfide generation in said oil-filled electrical apparatus on the basis of the detecting (a); and
(c) diagnosing a risk of anomaly occurrence in said oil-filled electrical apparatus on the basis of the evaluating (b),
wherein said specific compound comprises dibenzyl disulfide, a reaction product of a radical resulting from dibenzyl disulfide, or both, and di-tert-butyl-p-cresol, a reaction product of a radical resulting from di-tert-butyl-p-cresol, or both, or, di-tert-butyl-phenol a reaction product of a radical resulting from di-tert-butyl-phenol, or both.

2: The diagnosis method according to claim 1, wherein said specific compound comprises dibenzyl sulfide, and di-tert-butyl-p-cresol or di-tert-butyl-phenol.

3: The diagnosis method according to claim 1, wherein:
said specific compound comprises the reaction product of a radical resulting from dibenzyl disulfide; and
the reaction product of the radical resulting from said dibenzyl disulfide is at least one compound selected from a group consisting of benzaldehyde, benzyl alcohol, bibenzyl, dibenzyl sulfide and dibenzyl sulfoxide.

4: The diagnosis method according to claim 1, wherein said specific compound comprises contains a reaction product of a radical resulting from dibenzyl disulfide and a radical resulting from di-tert-butyl-p-cresol, or, a reaction product of a radical resulting from dibenzyl disulfide and a radical resulting from di-tert-butyl-phenol.

5: The diagnosis method according to claim 1, wherein:
the evaluating (b) is evaluating a possibility of copper sulfide generation in said oil-filled electrical apparatus is high in a case where said specific compound is detected in the detecting (a); and
the diagnosing (c) is diagnosing a risk of anomaly occurrence in said oil-filled electrical apparatus is high in a case where the possibility of copper sulfide generation is evaluated as high in the evaluating (b).

6: The diagnosis method according to claim 1, wherein copper sulfide generation in said oil-filled electrical apparatus is copper sulfide generation on a surface of an insulating paper.

7: A diagnosis apparatus, comprising:
a detection unit for detecting a specific compound contained in an insulating oil in an oil-filled electrical apparatus;
an evaluation unit for evaluating a possibility of copper sulfide generation in said oil-filled electrical apparatus on the basis of the detecting by said detection unit; and
a diagnosis unit for diagnosing a risk of anomaly occurrence in said oil-filled electrical apparatus on the basis of the evaluating by said evaluation unit,
wherein said specific compound comprises dibenzyl disulfide, a reaction product of a radical resulting from dibenzyl disulfide, or both, and di-tert-butyl-p-cresol, a reaction product of a radical resulting from di-tert-butyl-p-cresol, or both, or, di-tert-butyl-phenol a reaction product of a radical resulting from di-tert-butyl-phenol, or both.

8: The method of claim 1, which is suitable for evaluating a risk of copper sulfide generation in the oil filled electrical apparatus.
